(19) United States
(12) Patent Application Publication
(10) Pub. No.: US 2004/0224037 A1
(43) Pub. Date: Nov. 11, 2004

(54) Devastating treatment against hiv/aids with capsaicin

(76) Inventor: Gonzalo Romero-Matos, Saint Petersburg, FL (US)

Correspondence Address: GONZALO ROMERO M, APT #2, 455 39TH AVE NORTH, SAINT PETERSBURG, FL, 33703-6118, US

(21) Appl. No.: 10/805,843

(22) Filed: Mar. 22, 2004

Related U.S. Application Data
(60) Provisional application No. 60/465,760, filed on Apr. 28, 2003.

(51) Int. Cl. A61K 31/16; A61K 35/78

(52) U.S. Cl. 424/760; 514/625

(57) ABSTRACT

This method of medical treatment claims a use of Capsaicin (8-methyl-n-vanillyl-6-nonenamide), its derivatives, vanilloids and capsicum extract, to combat and control HIV (humans immunodeficiency virus) and aids (acquired immunodeficiency syndrome). An evaluation of a capsicum sp consumption of a long term aids survivors group permitted a definition of more efficacious ways to administer the substance. capsaicin intravenous and by subcutaneous or intramuscular administration at low concentration implemented by using infuses, it inhibits HIV replication and stimulates the production and proliferation of lymphocytes and cells nk. Also it acts as desinfectant in macrophages, and has a power as anticancer and antioxidant agent. Moreover has the property to control and annihilate common oportunistic illnesses related to HIV due to its triple antibiotic characteristics.

### CROSS-REFERENCE TO RELATED APPLICATIONS

This application is entitled to the benefit of Provisional Patent Application Ser. No. 60/465,760 filed 2003 Apr. 28. Otherwise, omit this section.

### BACKGROUND OF THE INVENTION

1. Field of the Invention

The invention has been found in the field of medical treatments of drugs, affecting biologically the human body.

2. Description of the Prior Arts

Capsaicin has been disclosed and taken as reference in this invention in agreement to Buck et al. (1986), The neuropharmacology of capsaicin: review of some recent observations, Pharmacological Reviews, vol 38, no 3, p. 180, like 8-methyl-n-vanillyl-6-nonenamide, (FIG. 1A). The substance is also known as (E)-N [(4-hydroxy-3-methoxyphenyl)-methyl]-8-methyl-6-nonenamide, according to U.S. Pat. No. 4,536,404, Bernstein (1985), p. 2, col 2, Method and composition for treating post-herpetic neuralgia. Capsaicin has also been identified as N-(3-methoxy-4-hydroxybenzyl)-8-methylnon-trans-6-enamide, by Monsereenusorn et al. (1982), p. 322, vol 10, Capsaicin-a literature survey, CRC Critical Reviews in Toxicology.

Synthetic Capsaicin, has been named as N-vanillylnonanamide by U.S. Pat. No. 5,461,075 (1995) O'Neill et al., Use of vanilloids for the prevention of lesions due to Herpes simplex infections, p. 7, col 9, and it has also been referenced by U.S. Pat. No. 5,431,914, (1995), Adekunle et al., p. 3, col 3, like nonivamide, (N-[(4-hydroxy-3-methoxyphenyl)-methyl]nonanamide).

Monsereenusorn et al. (1982), p. 322, has defined pharmaceutical compositions of four (4) natural derivatives of Capsaicin: Norhydrocapsaicin (7-methyl-octanoic acid vanillylamide); Dihydrocapsaicin (8-methylnonanoic acid vanillylamide); Homocapsaicin (9-methyldec-trans-7-enoic acid vanillylamide); and Homodihydrocapsaicin (9-methyl-decanoic acid vanillylamide). Chemical structures can be seen in FIGS. 1B, 1C, 1D and 1E.

The U.S. Pat. No. 5,461,075 (1995), O'Neill et al. (1995), p. 7, col 9 has also disclosed vanilloids compounds and are mentioned as more preferred substances to control Herpes simplex, The followings specific pharmaceutical compositions: N-vanillyl-9-octadecenamide; N-((4-(2-aminoethoxy)-3-methoxyphenyl)-methyl)-9Z-octadecenamide; N-(9Z-octadecenyl)-4-(2-aminoethoxy)-3-methoxyphenylacetamide; N-((4-(2-aminoethoxy)-3-methoxyphenyl)-methyl)-nonanamide; N-((4-(2-methyl-2-aminopropoxy)-3-methoxyphenyl)-methyl)-nonanamide; N-((4-(2-methyl-2-aminopropoxy)-3-methoxyphenyl)-methyl)-9Z-octadecenamide; n-(9Z-octadecenyl)-4-(2-amino-2-methylpropoxy)-3-methoxyphenylacetamide.

The prior arts to this invention are believed to be some references related to pharmacological treatments that have utilized capsaicin and its derivatives for a control, prevention and treatment of cutaneous viral illnesses. such treatments have been disclosed in the following patents of the United States. O'Neill et al., U.S. Pat. No. 5,461,075 (1995), p. 7, col 10, and Bernstein J. E, U.S. Pat. No. 4,536,404 (1985), pp. 1-2. Both patents propose a use of Capsaicin for herpes treatment in humans by topical applications. Additionally topical treatments of Capsaicin have been mentioned by Bernstein (1987), p. 352, in the article Capsaicin in the treatment of dermatologic disease and by Bernstein et al. (1987), p. 93, in the article treatment of chronic postherpetic neuralgia with topical Capsaicin.

Capsaicin for Herpes simplex by subcutaneous administration has been disclosed by Ljungdahl et al. (1986), p. 224, and by Harbour et al. (1983), p. 1492, both treatments were applied in mice with overdoses of 50 mg/kg bodyweight.

Specific bibliographic background that relates Human Immunodeficiency Virus (HIV) and Capsaicin is scarce however consistent. Thus, John Heinerman (1994), p. 94, refers to therapeutic benefits of the chilies, in a treatment of the Acquired Immunodeficiency Syndrome (AIDS). Heinerman also mentions an article written in the Journal of Orthomolecular Medicine by Calaph Timmerson (1990). According to this article, Calaph Timmerson, long term survivor of AIDS, attributed his relative recovery from aids as a result of a high diet of chilies during the last four years of suffering with this disease.

Likewise DeWitt (1998), pp. 2-4, made reference in “The healing powers of peppers”, an intent by Richard Quinn in a implementation of a rudimentary treatment of AIDS based on capsules of Capsicum Sp to control the disease in a group of patients in Minneapolis, USA.

### SUMMARY OF THE INVENTION

The present invention provides a method of medical treatment against HIV/AIDS with Capsaicin, a pungent principle in some species of the genus Capsicum Sp. This substance has been evaluated in this invention in relation to a therapy assumed by a group of long term aids survivors. Likewise Capsaicin has been studied herein according to its effect upon the immune and thermoregulatory systems in humans. It also is mentioned its power to control opportunistic diseases related to HIV/AIDS and induce antioxidant and anticancer effects.

The method includes treatments with Capsaicin by intravenous (iv.), and intramuscular or subcutaneous (im./sc.) administrations as alternatives more efficacious to control and break up the disease. in this invention are also considered treatments by digestive (ig.) administration and by topical application with Capsaicin.

### OBJECTS AND ADVANTAGES

The objects and advantages of the invention are: 1. To provide a multiple effect therapy against HIV/AIDS and related illnesses. 2. To provide a medication of high security for patients, whose side effects of Capsaicin administration are attenuated. 3. To provide a flexible treatment method implemented according to advance stage of the illness and physician criterial. 4. To provide a medication wherein doses and concentrations are clearly established and permissible. 5. To provide information basis for a further experimental clinical research.

### BRIEF DESCRIPTION OF THE DRAWINGS

For a greater understanding of the nature and objects of the invention a description, taken in connection with the attached drawings is used as refference:

FIG. 1A chemical structure of Capsaicin.

FIG. 1B chemical structure of norhydroCapsaicin.

FIG. 1C chemical structure of dihydroCapsaicin.

FIG. 1D chemical structure of homoCapsaicin.

FIG. 1E chemical structure of homodihyroCapsaicin.

FIG. 2 graphic of a function f (x)=1/x, where x is higher than 0, that describes a mathematical relation among a nervous desensitization in humans, with regard to dilutions of Capsaicin at iv. level.

FIG. 3 graphic of a function f (x)=1/x, where x is higher than 0, that describes a mathematical relation among a nervous desensitization in humans, with regard to dilutions of Capsaicin at im./sc. level.

FIG. 4 graphic of a function f (x)=1/x, where x is higher than 0, that describes a mathematical relation among a nervous desensitization in humans, with regard to dilutions of Capsaicin at ig. level.

### NUMBERS OF REFERENCE IN THE DRAWINGS

2A desensitization scale at iv. level (values 0-10).

2B dilutions of one part of Capsaicin in millions of parts of water (values 0-16).

2C persistent burning sensation in blood at 10 mcg/ml.

2D perceptible warmth sensation in blood at 1 mcg/ml.

3A desensitization scale at im./sc. level (values 0-100).

3B dilutions of one part of Capsaicin in millions of parts of water (values 0-1).

3C persistent burning sensation in tissues at 100 mcg/ml.

3D perceptible warmth sensation in tissues at 10 mcg/ml.

4A desensitization scale at ig. level (values 0-1000).

4B dilutions of one part of Capsaicin in millions of parts of water (values 0-0.1).

4C persistent burning sensation in stomach at 1 mg/ml.

4D perceptible warmth sensation in stomach at 0.1 mg/ml.

### DESCRIPTION OF THE DRAWINGS

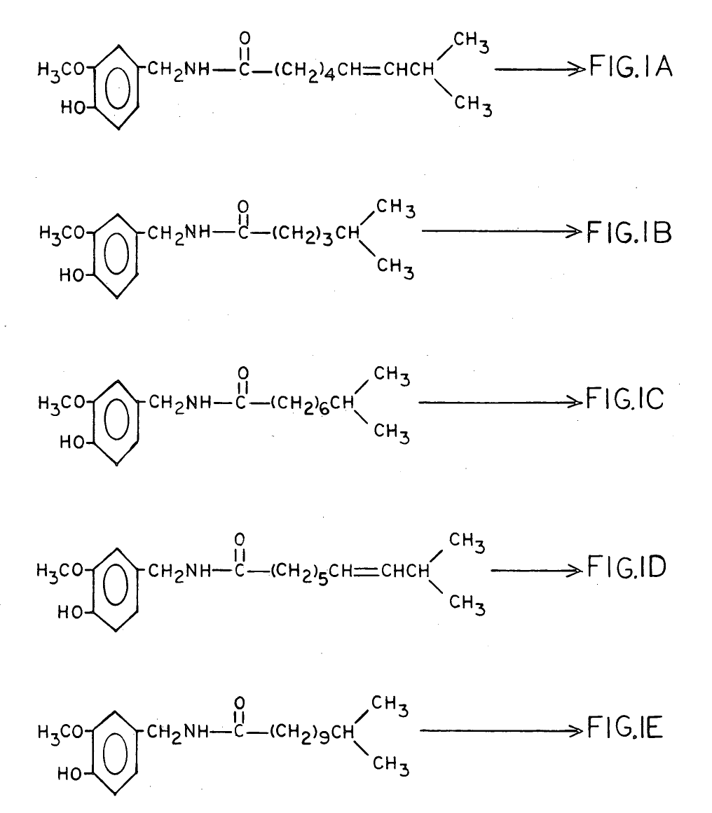

FIG. 1A corresponds to the chemical structure of the Capsaicin, FIG. 1B corresponds to the Norhydrocapsaicin, FIG. 1C corresponds to the Dihydrocapsaicin, FIG. 1D corresponds to the Homocapsaicin and FIG. 1E corresponds to the Homodihydrocapsaicin.

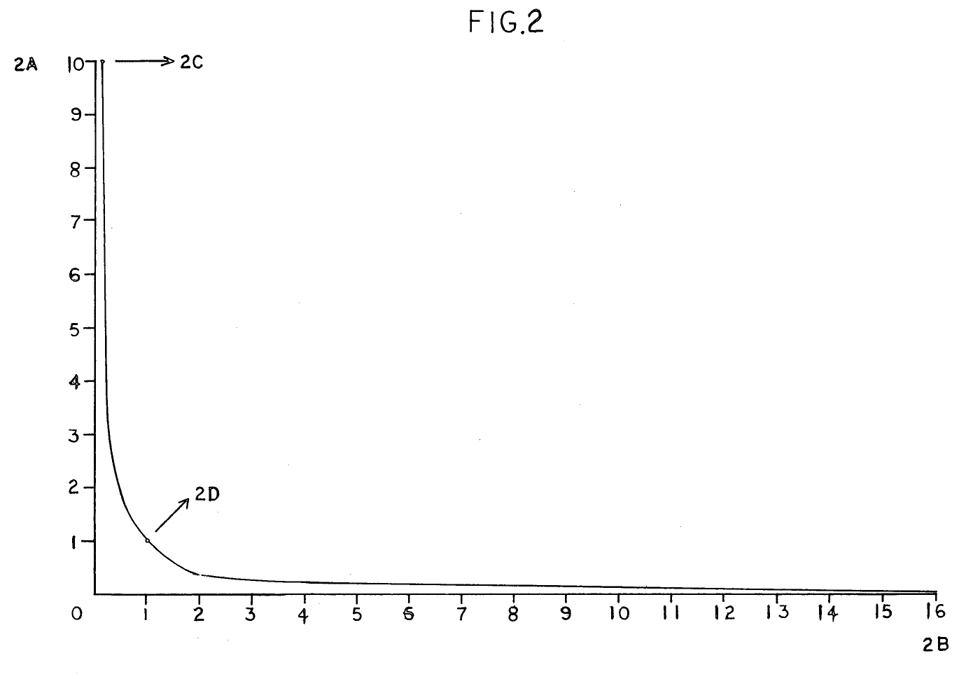

FIG. 2 shows a curve of the mathematical function that relates the nervous desensitization in humans with regard to the dilutions of Capsaicin at iv. level. The axis of the abscissa with values between 0 and 16, corresponds to the dilutions (2B) of one part of Capsaicin in millions of parts of water. The axis of the ordinate corresponds to the scale of desensitization (2A) with relative values between 0 and 10. The point (2C) corresponds to a concentration at 10 mcg/ml, and it represents to the Capsaicin concentration that provokes a persistent burning on tongue, or as assumed in this invention to the persistent burning sensation in blood. The point (2D) corresponds to a concentration at 1 mcg/ml, and it represents to the Capsaicin concentration that provokes a perceptible warmth on tongue, or as assumed in this invention to the perceptible warmth sensation in blood.

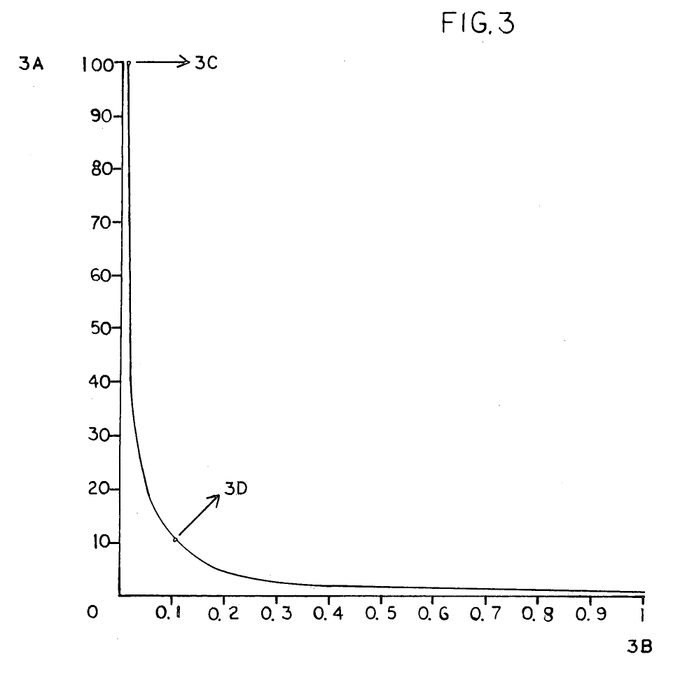

FIG. 3 shows a curve of the mathematical function that relates the nervous desensitization 1n humans with regard to the dilutions of Capsaicin at im./sc. level. The axis of the abscissa with values between 0 and 1, corresponds to the dilutions (3B) of one part of Capsaicin in millions of parts of water. The axis of the ordinate corresponds to the scale of desensitization (3A) with relative values between 0 and 100. The point (3C) corresponds to a concentration at 100 mcg/ml, and it represents to the concentration that provokes the persistent burning sensation in tissues. The point (3D) corresponds to a concentration at 10 mcg/ml, and it represents to the concentration of Capsaicin that provokes the perceptible warmth sensation in tissues.

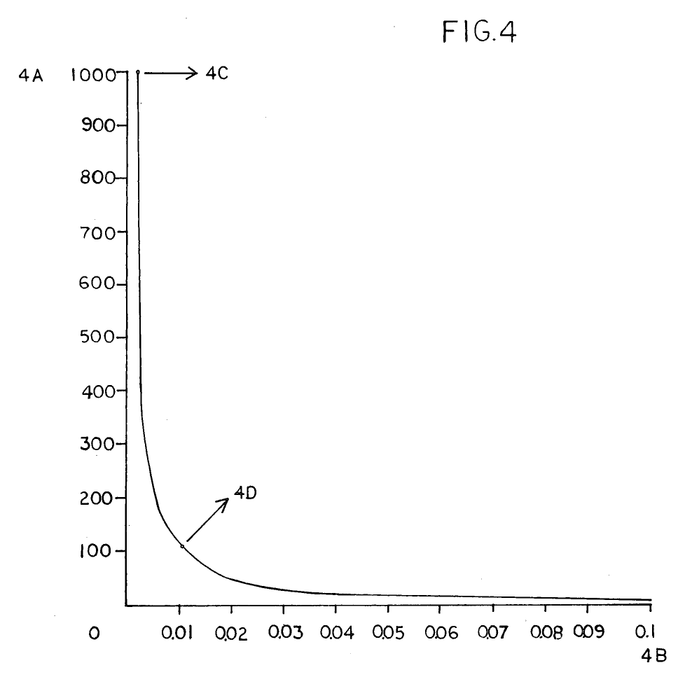

FIG. 4 shows a curve of the mathematical function that relates the nervous desensitization in humans with regard to the dilutions of Capsaicin at ig. level. the axis of the abscissa with values between 0 and 0.1, corresponds to the dilutions (4B) of one part of Capsaicin in millions of parts of water. The axis of the ordinate corresponds to the scale of desensitization (4A) with relative values between 0 and 1000. The point (4C) corresponds to a concentration at 1 mg/ml, and it represents to the concentration that provokes the persistent burning sensation in stomach. The point (4D) corresponds to a concentration at 0.1 mg/ml, and it represents to the concentration that provokes the perceptible warmth sensation in stomach.

Definition of Terms

Capsaicin when used herein includes natural and synthetic Capsaicin, derivatives, vanilloids and capsicum extract.

Natural Capsaicin derives from the fruits of solanaceae family and it is the pungent principle of the genus Capsicum sp. It is a stable, colorless, and odorless alkaloid, not very soluble in water, but very soluble in alcohol, greases and oils. Capsaicin and Capsaicinoids may be classified as acid amide derivatives of a phenol. With an addition of a methoxy group (OCH₃) to this phenol, ortho-methoxyphenyl is formed. An addition of a methylene group (CH₂) in the para position to ortho-methoxyphenyl it produces vanillyl. Capsicum fruits contain between 0.1 to 1% of Capsaicin.

Synthetic Capsaicin is a Capsaicin substitute and other synonyms used are nonanoic acid vanillylamide, nonylvanylamide, pelargonic acid vanillylamide, pseudoCapsaicin, N-vanillylpelargonamide and N-(4-hydroxy-3-methoxybenzyl) nonanamide.

Derivatives are vanillyl compounds as Capsaicin with similar action like Capsaicin but having it different degree of pungency. They form part of a complex of compounds called Capsaicinoids.

Vanilloids are phytochemical compounds that usually contain vanillyl moiety or analog structure as Capsaicin and they have antiviral properties.

Capsicum extract infuse may be a purified version of an infuse prepared from fresh plant tissues of any variety of the genus capsicum. Such an infuse may be elaborated by mixing capsicum extract with a pharmaceutical acceptable carrier to be injected by iv. and im./sc. treatments.

Level is considered a location in a particular system or part in the human organism responding at a different effect toward Capsaicin. It is related at a specific route of administration such as iv., im./sc., and ig.

Concentration of Capsaicin is a relative content of Capsaicin of a solution, mixture or dispersion that is expressed in this invention in mcg/ml and mg/ml. It may be taken from any point of the function in FIGS. 2, 3 and 4.

Dilution of Capsaicin is a variable of abscissa in the FIGS. 2, 3 and 4 in this invention that permit to make less or more concentrated Capsaicin by mixing with water. It is measured in one part of Capsaicin in millions of parts of water and may be transformed in units of concentration.

Primary afferent neurons are nervous fibers whose function is to receive and transmit information from an internal and external environment and thereby contribute to maintain homeostasis. They may be divided in a-type and b-type neurons and can be classified in thick myelinated, thin myelinated and thin unmyelinated (c-fibers).

Intrinsic origen nervous fibers are nerves of short distance, local reflex, insensitive to Capsaicin and predominantly founded in the digestive system. Extrinsic fibers are nerves of long distance, extensive effect in the organism, very sensitive to Capsaicin and predominantly founded in the circulatory system.

Neuropeptide is an endogenous peptide that influences neural activity or functioning. Sp or p(sp) is an undecapeptide of amino acid sequence arg-pro-lys-pro-gln-gln-phe-phe-gly-leu-met-nh2, which it has been identified in the central and peripheral nervous system.

Capsaicin desensitization is meant a typical feature of Capsaicin-induced stimulation of primary afferent neurons wherein excitation soon subsides and the neurons become unresponsive to further applications of the drug.

Perceptible warmth and persistent burning are Capsaicin sensations on tongue appreciated by nelson (1910), to a concentration at 1 mcg/ml (1 ppm) and at 10 mcg/ml (10 ppm) respectively, which they are assumed in this invention to affect the circulatory system of the human organism under same appreciation.

Desensitization scale is a variable of ordinate in FIGS. 2, 3 and 4 used to represent a relative degree or interval of nervous desensitization and it is defined according to values ranging between 1 to 1000. Perceptible warmth has a value of 1 at iv. level, 10 at im./sc. level and 100 at ig. level. Persistent burning has a value of 10 at iv. level, 100 at im./sc. level and 1000 at ig. level.

Scoville units value is the dilution in units or parts of water at which the pungency of one unit or part of Capsaicin can be detected by a trained tester.

AIDS in this invention is considered a disease of the human immune system that is caused by infection with HIV, and it is characterized cytologically by reduction in the numbers of CD4-bearing helper T cells. Although the relation between agent and disease is not absolute and definitive it is possible to affirm that AIDS is viral in origin.

HIV is any of a group of retroviruses that infect and destroy helper T cells of the immune system causing a marked reduction in their numbers. It also can infect macrophages. From this group, have been classified two variants identified as HIV-1 and HIV-2. A third variant of HIV called HIV-0 rather belongs to a subgroup of HIV-1.

CD4 receptors is a large glycoprotein located on the surface of helper t cells that is a receptor for HIV. CD8 receptors are located on the surface of killer T cells.

Lymphocyte is any of weakly motile cells originating from stem cells and differentiating in lymphoid tissue as of the thymus or bone marrow.

Macrophage is a phagocitic tissue cell of the reticuloendothelial system fixed or freely mobile, which it is derived from a monocyte, and it functions in the protection of the body against infection and noxious substances.

B-endorphin is a group of protein with potent analgesic properties that occur naturally in the brain.

Proliferation of macrophage denotes a macrophage which is capable of further clonal division. Normally macrophage is a terminally differentiated cell of immune system incapable of further division.

Polyamine is any of a group of aliphatic straight chain amines derived biosynthetically from amino acids. Polyamine analog is meant an organic cation structurally similar but non-identical to naturally-ocurring polyamines such as spermine and/or spermidine (taken of foreign patent number WO 9921542, of McGrath, 1999, p. 10, Methods for modulating macrophage proliferation using polyamine analogs).

Preferred agents for modulation of macrophages according to foreign patent number WO 9921542, of McGrath, 1999, p. 8, incorporated herein by reference are polyamine analogs specially 1,11-bis(ethyl)norspermine; 1,8-bis(ethyl)spermidine (BES); 1,12-bis(ethyl)spermine (BES; DESPM (N¹,N¹²-diethylspermine); 1,11-bis(ethylamino)-4,8-diazaundecane (BE-3-3-3); 1,14-bis(ethylamino)-5,10-diazatetradecane (BE-4-4-4) (diethylhomospermine, N¹,N¹⁴-diethylhomospermine; dehop or dehspm); diethylnospermine (denop); 1,19-bis(ethylamino)-5,10,15-triazanonadecane (BE-4-4-4-4); n-ethyl-n′-(2-(3′-ethylamino-propylamino methyl)-cis-cyclopropylmethyl)-propane 1,3-diamine tetrahydrochloride (SL-11037); N-ethyl-N′-(2-(3′-ethylamino-propylamino methyl)-trans-cyclobutylmethyl)-propane 1,3-diamine tetrahydrochloride (SL-11038); N-ethyl-N′-(2-(3′-ethylamino-propylamino methyl)-trans-cyclopropylmethyl)-propane 1,3-diamine tetrahydrochloride (SL-11044; and N′-bis (3-ethylaminopropyl)-cis-but-2-ene-1,4-diamine tetrahydrochloride (SL-11047).

Exogenous pyrogens agents are microorganisms or agents whose products, are recognized as foreign by the immune system cells. The exogenous pyrogens are capable of inducing pyrogenic cytokines, called endogenous pyrogens, such as: interleukins (IL-1b), (IL-6), tumor necrosis factor (TNF-α), and interferon (INF-a), Blatteis (1998), pp. 178-179.

Hypothermia is generally considered 1n humans, when a core temperature is less than 35 degrees centigrade (95 deg F). A rank between 34 and 35 degrees centigrade (93.2 to 95 deg F), is considered lightly hypothermic; 30 to 34 degrees centigrade (86 to 93.2 deg F), is considered moderately hypothermic; and less than 30 degrees centigrade (86 deg F), severely hypothermic, Mercer J. B, 1998, p. 246.

Skin temperature average in human adult man, in a thermoneutral environmental, without wind and low humidity is about 33 degrees centigrade degrees F), physiology and pathophysiology of the temperature regulation, Blatteis C, (1998), chapter 2, entitle body temperature, p. 17, table 1.

### DETAILED DESCRIPTION

Some factors that make to HIV a pathogen highly harmful and complex for the bearers of the illness have fully been established. The virus can grow in areas of the human body, where the immune system has a limited access, such as the central nervous system. It can be integrated to the genome of the cells of the body, where it can remain latent and unaffected for long time periods. It also can alter its antigens to generate mutations and infect and suppress the immune system causing indirect harm to the organism and developing other infections.

Despite such critical pathology of AIDS, a treatment or therapy can be an alternative to counteract the virus and its related diseases. For this reason, the therapy assumed by Calaph Timmerson, long-term AIDS survivor, requires a careful evaluation. In 1990, 1immerson wrote the article for the journal of orthomolecular medicine, on the therapy essentially defined in the consumption of chilies and other piquant foods, during four years of suffering the illness. According to this article, Timmerson's lymphocytes quantified through a test T-4, decreased to near of 50 per cubic millimeter (cm) of blood, p. 25.

HIV-negative patients have between 500 and 1,200 per cm. Ten (10) years after of suffering the illness, he expressed to be found in good physic condition, and manifesting himself a great reduction in the secondary illnesses. Nevertheless, so much in timmerson, as in a group of persons related to him, that assumed the same diet (high in piquant foods), was noted a constant decrease of lymphocytes in the immune system of theirs organisms.

Capsaicin iv. administration, or by im./sc. administration has a hypothetic capacity to stimulate a production and proliferation of lymphocytes, through a release of neuropeptides, such as a substance p(sp). This feasible stimulation does not happen by ig. administration because nervous fibers in the gastrointestinal tract have a local effect of intrinsic origen, in a release of the substance p(sp). According to Franco et al. (1979), p. 62, in intestines of guinea pigs exist a predominance of nervous fibers of short distance along the intestine and of local reflex in the organism. On the other hand, Holzer et al. (1980), p. 303, does mention of a predominant quantity of intrinsic fibers located in the gastrointestinal tract. In this same study, they show that, pretreatment with Capsaicin in rats, did not have effect upon the substance p(sp) at intestinal level, which it indicates that, the nervous fibers of intrinsic character are insensitive to the Capsaicin.

Effect Upon the Immune System

after Jancso's death in 1966, studies of Janos Szolcsanyi and aurelia Jancso-Gabor revealed that, most of Capsaicin biological effects result from initial arousal of certain sensory neurons, that is followed by a prolonged period of desensitization to physicochemical stimulus (Buck et al., 1986, p. 180).

Afterwards, intense investigations were carried out and they showed that, by effect of the Capsaicin, the substance p(sp) is released 1n sensory neurons. Buck et al. in 1986, p. 222, did a review of a profuse bibliographic reference to this respect. monsereenusorn et al. (1982), p. 332, shows a summary of different neurals structures where it was detected the release of the substance p(sp) caused by treatments with Capsaicin. They were spinal cord, dorsal horn, substantia nigra, trigeminal nucleus caudalis, substantia gelatinosa, sciatic nerve, saphenous nerve, sensory of the eye, and other. The substance p(sp), is part of a neuropeptides family called tachykns.

Holzer (1991), p. 146, refers some markers of Capsaicin-sensitive primary afferent neurons. These markers include a number of peptides that are released such as substance p(sp) by Capsaicin. They are neurokinin a, calcitonin gene-related peptide (cgrp), galanin, vasoactive intestinal polypeptide, and somatostatin.

The substance p(sp) is recognized for t lymphocytes, as was shown by Payan et al. (1984), p. 1532, in their evaluation of existing interaction among the substance p (sp) and human blood t-lymphocytes, quantified by a fluorescence-detection flow cytometry. This evaluation manifested a specificity of the substance p(sp) and h (sp) upon human t-lymphocytes, according to experiments carried out by them in 1983.

In agreement to McGills et al. (1990), p. 92, the substance p(sp) can alter a proliferative and physiological response of lymphocytes and macrophages. Those effects are mediated by a specific high affinity of receptors p(sp), which they have been characterized and identified extensively, so much in limphocytes and macrophages, from a point of view kinetical and biochemical.

In the summary of a study “Review modulation of the immune response by tachykinins”, elaborated by Eglezos et al. (1991), p. 291, it shows a description of the effects of tachykinins release: stimulate the proliferation of lymphocytes; increase a release of cytokines, including interferon(IFN-gamma), tumor necrosis factor (TNF-alpha), interleukins (IL-1 and IL-6) of mononuclear cells and macrophages; enhance immunoglobulin secretion, and affecT cellular chemotaxis and phagocytosis.

Operation of these processes previously defined, is feasible, through the treatment with Capsaicin, that can stimulate a production of new lymphocytes by the thymus. According to Eglezos et al. (1991), p. 286, the thymus is innervated by afferent nerves containing tachykinins as, p(sp) and cgrp. Those nerves, have fibers in contact with all components of the thymus and they have been associated with populations of lymphocytes and masT cells.

In patients with HIV, great quantities of t lymphocytes (helpers), are contaminated and inactivated by an affinity of the HIV upon CD4 specific-receptors. The proliferation of this type of lymphocytes is crucial for a development of immune mechanisms.

According to Panerai et al. (1983), p. 825, the Capsaicin also exerts a release or decrease of b-endorphin concentrations in the brain tissues. In experiments developed in rats, b-endorphin concentration in the hypothalamus, measured at ng/mg of protein, decreased, during 3,5,7 and 15 days, thereafter they gave the Capsaicin administration. The obtaining of results was done in base to tissues extraction in the hypothalamus and other regions of the brain. It showed that, the Capsaicin is capable to release b-endorphin in rats brain. On the other hand, gilman et al. (1982), p. 4226, demonstrated that, the b-endorphin increases, the proliferative response of lymphocytes. They suggest that b-endorphin has a specific effect on cellular components of the immune system, such as t-lymphocytes.

The b-endorphin, as well increases a natural cytotoxicity, induced by killer (nk) cells. It was shown for Mathews et al. (1983), p. 1658. According to them, activity of nk cells was significantly increased by b-endorphin in 30.5% with a deviation of more or less 11.5%. Likewise, they expressed that, a role of these neuropeptides is relatively unknown, nevertheless receptors of b-endorphin have been detected in phagocytes and human lymphocytes.

Jancso and Jancso-Gabor established in 1947 and 1959, that the Capsaicin as well as others classes of injuries and burns, caused a histamine release, promoting phagocytosis, and transforming, endothelial cells of capillaries into phagocytes. These experiments of Jacnso are mentioned for Issekutz et al. (1950), p. 321, and by Monsereenusorn et al. (1982), p. 323. Likewise, experiments of Holzer et al. (1981), p. 1099, confirm it previously expressed, showing the fact that, treatment with Capsaicin in rats provoked an increment of histamine and 5-hydroxitryptamine.

On the other hand, Johnson et al. (1973), p. 1253, demonstrated that neuropeptides provoked the histamine release, 1n suspensions of masT cells in rats. Substance p (sp) and polisteskinin were the neuropeptides more powerful, stimulating liberation over a 50%. Less powerful were: bradykinin, kallidin and methionyl-lysyl-bradykinin.

In 1983, Skofitsch et al., p. 153, evaluated with higher precision some Capsaicin, histamine, and substance p(sp) acting mechanisms. They demonstrated that, the histamine release is provoked for the substance p(sp), and not by the Capsaicin directly. Bar-shavit et al. (1980), p. 1145, also alleges that the undecapeptide substance p (sp) as well stimulates phagocytosis in macrophages of mice and 1n human leucocytes. It was confirmed for Hartung et al. (1983), p. 301, with guinea pig peritoneal macrophages. The substance p(sp) activated such macrophages, inducing a liberation of reactive oxygen species.

Macrophages are capable to absorb efficiently the Capsaicin. In Joe and Lokesh experiments (1994), p. 259, with rats peritoneal macrophages, they monitored an absorption of different spicy substances from macrophages, to evaluate if a internalization of those components is essential to reduce reactive oxygen species. Macrophages internalized the spicy substances efficiently and the Capsaicin was absorbed between concentrations (1-10 mcm), with a 76 to 82% of efficiency.

Capsaicin absorbed by HIV contaminated macrophages and lymphocytes may act as disinfectant agent against HIV virus in cellular vacuoles and cytoplasmic content. It is known that certain strains of the virus have a greater affinity for macrophages than for lymphocytes becoming such cells in elements of propagation of HIV. Internalized Capsaicin in macrophages may destroy and denature HIV and help to inhibit oxidation products. In addition macrophages carrying Capsaicin can be attracted to the center of HIV infections destroying focus of the virus.

A relative proliferation of macrophages produced by Capsaicin must be analyzed according to 1. Capsaicin in this invention is employed at low doses and concentrations. 2. After repetitive Capsaicin doses, substance p(sp) and histamine is released and it get a decreasing effect on macrophages and phagocytosis. 3. Capsaicin being able to abort HIV infection inside and outside of cells of the immune system will provide a way of delaying development of macrophages-associated diseases. 4. Macrophages without Capsaicin treatment is a factor of HIV propagation, instead macrophages under Capsaicin treatment become in a factor of HIV annihilating.

Effect Upon Opportunistic Illnesses

Studies of Cichewicz et al. (1996), p. 61, about antimicrobial properties of the chilies confirmed an inhibitor effect of capsicum sp in a growth of the following pathogens: Bacillus cereus, Clostridium sporogenes, Bacillus subtilis, Clostridium tetani (cause of the tetanus) and, Streptococcus pyogenes (cause of cutaneous and systemic infections). Some trials carried out using only Capsaicinoids (Capsaicin and dihydroCapsaicin), they did not show activity in the following pathogens: Candida albicans, Salmonella typhimurium, Staphylococcus aureus, Escherichia coli, Streptococcus pyogenes, Clostridium sporogenes, Bacillus cereus, and Bacillus subtilis, p. 63. It suggests, according to the authors of the study that, species and varieties of Capsicum sp played a medicinal role in some mayan sanatorium practices, p. 68.

Foreign patent number JP10120593 (Masaru H, 1996), antifungal agent for external use, includes to the Capsaicin to control superficial mycosis, and it mentions that Capsaicin is a tachykinins releasing agent, (see abstract of the invention). Likewise the U.S. Pat. No. 6,063,381 (2000), of Staggs J J, therapeutics uses of pungent botanicals and their related compounds, shows that dermatomycoses caused by Tinea pedis, Tinea capitis, Tinea corporis, Tinea cruris and Candida may be completely healed after a single treatment with capsicum extract or another pungent botanical species. (p. 4, col 5). This patent also mentions that capsicum and related families provide an important tool in the systemic treatment of deep tissue mycosis. (p. 8, col 14).

In addition to the antibacterial and antifungicidal properties mentioned, Capsaicin possesses antiviral properties as well. Jungdahl et al. (1986), p. 223, proved that Capsaicin injected by sc. Administration, reduced mortality in rats infected with herpes simplex. On the other hand, in the article “Capsaicin in the treatment of dermatologic disease”, Bernstein (1987), p. 352, reveals that the Capsaicin has been mainly used in a treatment of two cutaneous disorders: postherpetic neuralgia (phn) and psoriasis. These observations were confirmed afterwards by Bernstein et al. (1987), p. 93, with topic applications of Capsaicin in affected dermatome with herpes zoster.

The Capsaicin antimicrobial properties are found in direct relation with a its pungency degree. The Capsaicin pungency produces a heat, that placed in contact with pathogens lipoproteinic cellular membrane, originates a denaturation of membranes chemical structures. According to kauzmann, in a study titled the influence of the temperature upon the biological systems (1957), the proteins are the components most sensitive in the living systems, and an important factor that affects to them, is the temperature, which it is responsible by a protein thermal instability and denaturation. As it can be considered, the Capsaicin does not have specificity on any affinity upon certain types of microbes. This Capsaicin action wide spectrum, enables a wider opportunistic illnesses control in patients with HIV and AIDS.

According with Montagnier (2000), p. 153, when the level of t4 lymphocytes in HIV patients blood falls to between 500 and 200 per c.m., three pathologies often occur: candidiasis (caused by the fungus Candida albicans), shingles caused by the Varicella-zoster virus and leukoplakia of the tongue. Capsaicin treats candidiasis and shingles. A curative power of Capsaicin to control shingles or (phn) which it is a posterior and recurrent form of infection caused by Varicella-zoster virus has been mentioned by dewitt(1998). According with Timmerson (1990), p. 25, he reported in his article great reduction of opportunistic illnesses, despite of his level of lymphocytes (around 50 per cm).

Effect Upon the System of Thermoregulation

Injected Capsaicin by iv. or im./sc. administration produces among other phenomenons, a hypotension and a vasodilation in the circulatory system (Monsereenusorn et al. 1982), pp.323-324, from the generated heat by the pungency of the substance. This increased heat load is detected by the hypothalamus cooling center (Jancso et al. 1966), p. 364, which it sends messages to increase blood flow toward the organism surface, and to expand capillaries of the dermis. It permits, the heat to pass, from the body's core to the skin. That lost heat is reached by physic process of convection, conduction and radiation. By this way, is produced a known physiological phenomenon of sweating by consumption of chilies, gustatory sweating (Andrews, 1984, pp. 73-74).

This vasodilation caused by the Capsaicin conducts a temperature decrease or hypothermia, which it has a dependent intensity of an applied dose (Jancso et al. 1966, p. 364, Jancso-Gabor et al. 1970, p. 497).

It is considered hypothermia generally in humans, when a core temperature is less than 35 degrees centigrade (95 deg f). A drop of 2 degrees centigrade can provoke only some symptoms of sHIVering and a sensation of being cold.

HIV has been considered a virus type that manifests a persistence or latency in the attacked organisms. Some causes and mechanisms that determine the persistence of a virus, according to different biological situations implied, were analyzed by Frankel et al. (1982), pp. 362-365. They are 1. integration of the viral genome into the host 2. viruses that are nonantigenic and also certain viruses transported in lymphatic cells (some retrovirus). 3. viral affinity to neural tissues. 4. viruses where a balance, among virus infectivity and an interferon stimulating activity becomes a determinant factor, if, an infection is aborted or maintained. 5. apparition of defective interfering particles. 6. and viruses that it tends to mutate frequently in the organism by temperature cause (temperature sensitive mutants), with slow replication, to cause symptoms or to unchain the immune system response.

HIV fulfills the characteristics of Frankel et al. (1982), and so it as temperature sensitive mutant virus, and can be classified as “restrictive”, whose range of temperature oscillates between 36 and 41 degrees centigrade (96.8 to 105.8 deg f). Viruses classified as “permissive” have a range of temperature that oscillates between 30 and 35 degrees centigrade (86 to 95 deg f). Common cold is a type of permissive virus, due to a growth optimun temperature is manifested in the respiratory tract of the human organism. HIV optimum temperature range corresponds to the established range mentioned acording to the classification of Zinsser, 1972, p. 794, for the restrictive viruses. A provocation of a light hypothermia of 2 degrees centigrade it starts to generate HIV replication inhibition.

According to Black 1993, p. 500, viruses such as influenza, parainfluenza, and rhinoviruses, are sensitive to the temperature. When a patient is affected with fever of 1 or 2 degrees centigrade, virus replication ability is harmed. A momentary vasodilation as it occurs with the treatment with Capsaicin increases the blood flow toward the skin to produce a necessary heat unload of the body. This mechanism generates a drastic effect in the restrictive virus temperature optimum range. The HIV inhibition replication is accentuated, due to that, the skin temperature is 4 to 5 degrees lesser than an internal temperature of the body.

A treatment for HIV by using hyperthermia can be as effective inhibiting HIV replication, as by using a light hypothermic treatment. The foreign patent number WO0203879 (2001) of Groth, Kelly, Westerbeck and Blick, Treatment of HIV using hyperthermia, mentions that, raising the core temperature of HIV positive patient and returning the core temperature of the patient to normal at least one time: 1. Eliminate or reduce viable HIV to an extent around three (3) months and 2. Result in an increase in the CD8% around one month after raising the core temperature.

According to the mentioned invention one of the more preferably embodiment it is when the core temperature can be raised to a temperature range between 41.8 to 42.2 degree centigrade (107.2 to 107.9 deg f), p. 4.

The Capsaicin as “alkaloid” can be included, in a classification of exogenous pyrogens agents, not as a microbial drug. According to Blatteis (1998), pp. 178-179, it is now established that, a fever and the correlation of the sharp phase not thermic are not induced directly by original pathogens agents, but by endogenous pyrogens or mediates (cytokines). The Capsaicin by its relative toxicity at low concentratrations, can be recognized for the human organism as an exogenous pyrogen agent which it has a capacity to stimulate pyrogenic cytokines. Also, a Capsaicin thermic effect, in this case, as exogenous pyrogen agent, it can be stimulating factor of interferon and others endogenous agents.

Antioxidant Effect

In a study of some Capsaicin congeners sensory effects, Szolcsanyi et al. 1975, p. 1878, define three (3) fundamental parts of the Capsaicin molecular structure: an acyl-amide link, an alkyl chain and an aromatic ring (phenolic group). Belitz et al. (1987), p. 175, reveals that antioxidants containing a phenolic group play a major role in food. According to Stasch et al. (1970), p. 410, the chilies possess antioxidant properties, because they contain ascorbic acid, tocopherols, flavonoid compounds and principles of pungency. Research on Capsicum firutescens influence, in frozen and stored meats leave no doubt that the chilies have such properties.

Also experiments of Joe et al. 1994, p. 255, demonstrated that 10 mcm of Capsaicin, from red pepper, completely inhibited a production of anions superoxides, peroxide of hydrogen and nitrites by macrophages in vitro, in comparison with others spicy substances. Higher concentrations of eugenol (from clove) and piperine (from pepper) were required to completely inhibit reactive oxygen species. Montagnier (2000), p. 185, mentions that in patients suffering from AIDS, is noted a significant antioxidant shortage and also an oxidation product increase, causing a fast enzyme collapse and a stress that can activate a cellular apoptosis. Such experiments of Joe et al (1994) finally show the Capsaicin benefits in the free radical inhibition.

Anticancer Effect

In an article of Zhang et al. (1993), p. 2341, they show that the Capsaicin inhibited a tobacco specific carcinogen [4-(methylnitrosamino)-1-(3-pyridyl)-1-butanone] nnk, present in the smoke of the tobacco. Gannett et al. (1990) demonstrated interestingly, that the Capsaicin is capable to inhibit an activity of enzyme (cyp2e1) of cytochrome p4502e1 in the liver, which it is responsible for a Capsaicin conversion to radical phenoxy.

In some conclusions, Surh et al. (1995), p. 1853, cite with certainty that, variations in the results on Capsaicin mutagenic activities, are attributed to inhibition or induction of cyp2e1 activity by modulators of this enzyme, affecting a compound metabolic activation to mutagenic species. Another conclusion of the same study was, that a balance between activation and detoxification, can be critical in determining of a dose related to a Capsaicin toxicity. At lower doses, toxic metabolites, are really removed by cellular nucleophiles or via another conjugation reactions that including sulfation and glucuronidation. If such defensive processes are saturated by high dosage of Capsaicin, toxic metabolites will accumulate and noxious effects will be manifest.

The U.S. Pat. No. 5,569,673 (Morre et al. 1986), p. 1, Capsaicinoid compounds as proliferation inhibitors, and foreign patent number WO2002067 (Morre D M and Morre J D, 2002), compositions based on vanilloid-catechin synergies for prevention and treatment of cancer, also evaluate some capacities of Capsaicin as inhibitor of the cancer. Capsaicin is found to inhibit growth at low concentration. Patents before mentioned are of particular interest due to that, HIV patients suffer of carcinogenic illnesses as the Sarcoma of Kaposi and Lymphomas.

Physiological Effects

Additionally, and according to U.S. Pat. No. 6,022,718, Iwai, et al. 2000, method of producing Capsaicin analogues, p. 2, col 1, are known various Capsaicin effects useful to living organisms such as an appetite promoting, salivation and intestinal peristalsis stimulating effect. It also is able to produce a vasodilating and vasoconstricting and a circulatory cholesterol level reducing effect. Likewise it can provoke an energy metabolism enhancing and a bioactive peptide release stimulating effect.

Immunization Mechanisms

In Non-allergic patients to Capsaicin it is possible to induce an immunization process against HIV. When low concentrations and doses of Capsaicin are administered in the blood, instead of a light toxicity or light allergic reaction, a prophylaxis is produced instead. Apparently Capsaicin and allergens follow a same pathway when the human organism reacts to such substances. According with Lunblad et al. (1987), p. 23, it is concluded that Capsaicin sensitive sensory nerves are of importance for a human cutaneous triple response reaction induced by allergen exposure. Thus, secondary release of mediators, as cgrp or tachykinins from sensory nerve branches may contribute to a flare and itching of this reaction.

The foreign patent number JP09249579 (Hiroshi N, 1996), treatment of HIV with antigen of pollen or IgE antibody thereto, mentions that intravenous injection of a pollen antigenic substance such as Cryptomeria japonica d or others species are capable of activating helper T cells or b cells to promote a proliferation thereof. In HIV patients helper T cells are extremely reduced. Thereby, the existence of the pollen antigenic substance or IgE antibody which reacts reversibly thereto is obstructive to HIV, (see abstract of the invention).

The heat load of Capsaicin generated by direct contact with HIV will produce denaturation and fragmentation of the virus. The foreign patent number WO 02/03879, of Groth et al. (2002) already mentioned, states that heat damage to nonviable HIV may also act as a vaccine, stimulating b-cells to form antibodies and may cause t-cell response to the specific HIV fragments or nonviable virus, further enhancing immunity, p. 6.

Test of Allergies

Before to an application of the treatment with Capsaicin by iv. or im./sc. administration or prior to a pretreatment, it is necessary to recognize, if a patient is sensitive or allergic to the Capsaicin and its derivatives. For this reason, an allergy test can prevent an anaphylaxis produced by the substance. A test called “radio-allergosorbent testing” (rast), can define itself as the most adequate, since a patient blood serum is combined with the substance, to determine if antibodies get reaction. Levels of immunoglobulins in blood over the normal suggest a hypersensitivity to the Capsaicin.

Pretreatment

In most of the experiments carried out in animals with Capsaicin by iv. administration, with moderate and high concentrations, it has been reported a manifestation of a triad of effects or Bezold-Jarisch reflex, hypotension, bradycardia and apnea, (Szolcsanyi et al. 1971, p. 260). Some doses and effects produced in animals by iv. treatments are:

1 0.56 mg/kg in male mice. effect: lethal in 50% of a tried animal population-LD50 (Glinsukon et al. 1980, p. 218).

2. 400 mcg/kg in cats. effect: higher post-hypotension and a second period of apnea (Porszasz et al. 1955, p. 62).

3. 200 mcg/kg in cats. effect: bradycardia, apnea and a tension reduction by 46 mmhg, in comparison with the previous experiment (Porszasz et al. 1955, p. 62).

4. 50 mcg/kg in cats and dogs. effect: fast fallen in blood pressure, a drop of the pulse at 108/min and 24/min, with subsequent tachycardia at 160/min; the pulse returns to the normal (Porszasz et al. 1957, p. 190).

5. 10 mcg/kg in dogs. effect: apnea, bradycardia, hypotension (Palecek et al. 1989, p. 1429).

6. 1 mcg or 3 mcg/kg in rats. effect: reproduced three phases, initial fall in blood pressure accompanied by a fall in heart rate, followed by an increment both, and subsequently a light and sustained increment of the blood pressure (Chahl et al. 1987, p. 414).

As it can be observed, doses of Capsaicin among 3 and 400 mcg/kg reproduce the Bezold-Jarisch reflex, with certain attenuation of the effects, when the doses get lower. Chahl et al. (1987), p. 415, tried with different methods to attenuate the Bezold-Jarisch reflex (vagal section, atropine injections, hexamethonium, propranolol, phentolamine, and pretreatment with Capsaicin) also, Makara et al. (1967), pp. 39-42, studied the phenomenon, experiencing with Capsaicins pretreatment, prior to intravenous treatment with the same one substance. The results were the following:

1. 1 mcg of injected Capsaicin in rats with previous anesthesia, and a Capsaicin's pretreatment of 100 mg during four days. effect: normal blood pressure and rate of the heart. (Chahl et al. 1987, p. 415).

2. 50 mcg of injected Capsaicin with slow infusion, during a period of 12 mins, with previous anesthesia and Capsaicin's pretreatment of 100 mg during four days. effect: normal blood pressure and rate of the heart (Chahl et al. 1987, p. 416).

3. 18 mcg/kg of injected Capsaicin in rats, with previous anesthesia and a Capsaicin's pretreatment at 4,8,16 and 200 mg/kg each 12 hours. effect: significant inhibition of the Bezold-Jarisch reflex (Makara et al. 1967, pp. 39-42).

It is shown that, Capsaicin pretreatment applied it before to iv. treatment attenuates the effects of the same substance. a method used by Chahl et al. (1987) for the pretreatment was that of Morton et al. (1980), p. 272. to establish the Capsaicin pretreatment in this invention, will be applied by im./sc. injection without anesthesia, to a proportion 10 times over a first iv. dose adopted at 1 mcg/kg, during three days in a same area of injection for applying the iv. treatment. Thus it will be applied: 2 mcg/kg (first day), 3 mcg/kg (second day), and 5 mcg/kg (third day).

Intravenous Treatment

Capsaicin iv. concentration and the nervous desensitization factor to the Capsaicin are determinants. From these variables depend the substance p(sp) release, and the adequated stimulation of the immune system. In a study of a Capsaicin chemical isolation, done with the purpose to determine the molecular structure of the substance and its properties, E. K Nelson (1910), p. 420, indicated: a drop of a solution of Capsaicin containing one part in 100,000 parts, causes a persistent burning on the tongue. A drop of a solution of Capsaicin containing one part in 1,000,000 parts, produces perceptible warmth. Nine years later in 1919, Nelson clarified the formula about its structure and resolved its synthesis (nelson, 1919, 1920, 1923; Porszasz 1955; Crombie et al. 1955). Such indicators permit a construction of a function (FIG. 2) between these Capsaicin concentrations and the desensitization of the body to the substance. The dilution of one part of Capsaicin in 1,000,000 parts of water, denotes a concentration at 1 mcg/ml and the dilution of one part of Capsaicin in 100,000 parts of water, denotes a concentration at 10 mcg/ml.

The tongue is a part of the body extremely sensitive containing it abundance of nerve fibers. For such reason the sensations of nelson, perceptible warmth (2d) and persistent burning (2c)on the tongue are assumed for the blood. Nagy et al. (1982), p. 3149, mentions that sp-nervous fibers are densely packed in bundles around basal regions of the taste buds in rat tongue. On the other hand Karrer et al, 1991, mentions that, additionally to the four classes of taste found on human tongue, there is another chemical detection system found in the oral cavity, as well as the nasal cavity, the respiratory tract, the eye, and perhaps even in nonmucous membrane skin. This system detects chemical irritants, and generally induces protective reflexes, p. 757. As e.g of this chemical detection system it is possible to mention about the Bezold-Jarisch reflex manifested in humans.

Capsaicin concentration can be studied in relation to the Capsaicin nervous desensitization. Accordingly it is possible to assume these indicators of concentration above mentioned in function of the scale (2a), where: the number 1 value is equivalent to the sensation (2d) and the number 10 value is equivalent to the sensation (2c). These values can be plotted to determine and study the characteristics of the existing function, among the values of the scale of desensitization from the number 1 value until 10, with regard to the dilutions (2b) of one part of Capsaicin in millions of parts of water.

An image of the function (FIG. 2), is a hyperbola without limit up apparently, where f(x)=1/x and x is higher than 0.1n the function a central point can be observed, it is given in the concentration at 1 mcg/ml and the sensation (2d). This point can be considered as a limit which finishes with a light desensitization to the substance, and begins a progressive and accelerated desensitization process to the Capsaicin. After this point, the Capsaicin concentrations notably increase by a 50% for each 200% of increment in the desensitization. Olmested (1968), p. 139 shows a typical unbounded mathematical function, where such function can be observed.

According to Holzer (1991), p. 150, the most typical feature of Capsaicin-induced stimulation of primary afferent neurons is that excitation soon subsides and the neurons become unresponsive to further applications of the drug. By this reason the scales in the FIGS. 2, 3, and 4 are measured in agreement to the nervous desensitization process. Likewise and according to Bernstein (1987), p. 96, a tachyphylaxis usually develops with repeated applications of Capsaicin. This phenomenon produces a diminished response to later increments in a sequence of Capsaicin administration. When concentrations and doses of Capsaicin increase, the desensitization process gets higher values, and the release of substance p(sp) become stronger and a reserve of this substance start to diminish. For these reasons before mentioned, the treatment of Capsaicin against HIV is needed to increase in concentrations until the limit (8 mcg/ml), following the function of the FIG. 2.

A intravenous treatment by applying concentrations below 1 mcg/ml does not guarantee an adequate release of neuropeptides because, the process of desensitization in this section of the function is not significant (just affects 10% of the process below 10 mcg/ml). Likewise, a treatment over 10 mcg/ml (persistent burning) is considered a beginning of nerve deterioration.

A medication which contains Capsaicin for iv. treatment, will depend on a sensitivity of a test of high performance liquid chromatography (hplc). This test is sensitive to two (2) parts per million (2 mcg/ml). For this reason the concentrations of the iv. treatment will oscillate between 2 and 8 mcg/ml of Capsaicin.

Subcutaneous or Intramuscular Treatment

According to Glinsukon et al. (1980), p. 216, a Capsaicin toxicity effect measured by LD50 in mice is similar when the substance is administered by the following routes, sc., im., and intraperitoneal (ip.).

Im. and sc. treatment concentrations were determined in base to the function f(x)=1/x, where x is higher than 0. This function is observed in the FIG. 3. Concentrations vary between 10 mcg/ml and 80 mcg/ml. Experiments of Simone et al. (1989), p. 99, can be taken by reference to evaluate an effect of Capsaicin in humans after intradermal injection. In this experiment, using doses between 10 and 100 mcg the area of hyperalgesia grew to reach a maximum within 5 and 7 min following the injection and gradually decreased, disappearing it within 15 and 137 min, respectively.

Topical Treatment

Capsaicin topical medication has been studied by Adekunle et al. (1995), p. 3, col 4, in U.S. Pat. No. 5,431,914, Method of treating an internal condition by external application of Capsaicin without the need for systemic absorption. In such method of particular interest the medication is applied to the skin and acts at a site distant. External applications of Capsaicin stimulate sensitive primary afferent nerves in the skin which lead to spinal cord segments affecting certain internal organs. This method may be applied to the area of the skin affecting the thymus to stimulate proliferation of lymphocytes in combination with systemic effect of Capsaicin absorption. Nevertheless it is a matter that requires further investigation. Same doses and concentrations as in im./sc. Treatment may be used by rubbing a cream or solution or utilizing patches.

Velocity of Administration

The velocity of administration of the Capsaicin, has a great importance in an attenuation of the triad of effects produced by the substance (bradycardia, hypotension and apnea). In a experiment of Schertel et al. (1986), p. 1237, they tried to evaluate some effects of the Capsaicin, in the respiratory system of dogs, previously submitted to get anesthesia with chloralose, at 80-100 mg/kg. a Capsaicin injected in a rank of doses at 0.5-20 mcg/kg induced equivalent of apnea, followed by a fast superficial respiration. A variation of the Capsaicin administration speed to a rate at 10-20 mcg/kg/min, provoked a fast superficial respiration, but without apnea. This evidenced that, varying the Capsaicin velocity administration contributed to attenuate the triad of effects. For a practical application of the Capsaicin administration speed, first half of doses will be injected at least to a rate at 1 ml/min, and remainders of the doses at a rate of 1 ml/30 seg, for iv. and im./sc. treatments.

Digestive Treatment

Some doses applied in some experiments which they have been tried before, define a framework of reference about the effect in the digestive system in humans and animals, toward the Capsaicin:

1. Capsaicin increases a gastric acid secretory rate, significantly and progressively, with increasing doses of Capsaicin, in rats. Acid secretory response reached a maximum Capsaicin dose at 1000 mcg/kg. higher doses caused a reduction of acid output (Limlomwongse et al. 1979, pp. 773-775).

2. capsicum solution elaborated with 3grs of dry powdered capsicum in 30 ml of water (1000 mcg/ml, with 1% Capsaicin), generated mucosal edema, hyperemia and hemorrhagic spots in the stomach in humans (Viranuvatti et al. 1972, pp. 225-226).

3. 1.25 grs of chilies in 30 ml of water (416 mcg/ml, with 1% Capsaicin), caused a moderated hyperemia according to gastroscopic diagnosis in humans (Schneider et al. 1956, p. 727).

4. 140 mcg/ml of Capsaicin caused an inhibitory effect of intestinal glucose absorption in rats in vitro (monsereenusorn et al. 1979, p. 393).

These experiments clearly indicate two points or limits in the function of desensitization to the Capsaicin, with regard to the concentration of the substance showed in the FIG. 4. These points are 1000 mcg/ml (1:1,000) and 100 mcg/ml (1:10,000). The concentration at 1000 mcg/ml, corresponds to the sensation (4c) in stomach, and the concentration at 100 mcg/ml, corresponds to the sensation (4d) in stomach.

For the digestive treatment with Capsaicin, it will be applied concentrations oscillating in a range between 100 and 450 mcg/gr of capsicum sp.

Potency of Capsaicin

An action or effect of the Capsaicin at its three different levels has its equivalent values of concentrations and sensations, according to the FIGS. 2, 3, and 4, and the following statements:

1. The sensation (2d) perceptible warmth in blood for iv. level, at the concentration of one part of Capsaicin in 1,000,000 parts of water (1 mcg/ml) is equivalent to the sensation (3d) for im./sc. level at one part of Capsaicin in 100,000 parts of water (10 mcg/ml), and it is equivalent to the sensation (4d) for ig. level, at one part of Capsaicin in 10,000 parts of water (100 mcg/ml).

2. The sensation (2c) persistent burning in blood for iv. level, at the concentration of one part of Capsaicin in 100,000 parts of water (10 mcg/ml) is equivalent to the sensation (3c) for im./sc. level at one part of Capsaicin in 10,000 parts of water (100 mcg/ml), and it is equivalent to the sensation (4c) for ig. level, at one part of Capsaicin in 1,000 parts of water (1000 mcg/ml).

This reveals that an action of the Capsaicin for iv. level, it has 10 times more potency than the action of the substance for im./sc. level, and 100 times more potency than an action of the substance for ig. level.

Glinsukon et al. (1980), p. 218, scheme it what was before mentioned, proportions of Capsaicin average letal doses (LD50) in mice are adjusted in approximated way to proportions of concentrations referred according to the potency of Capsaicin:

| | GLINSUKON EXP | PROPORTIONS | DEVASTATING TREAT | PROPORTIONS |
|---|---|---|---|---|
| iv. adm | 0.5 mg/kg | 1 | 1 mcg/ml (10 mcg/ml) | 1 |
| sc. adm | 8.5 mg/kg | 17:1 | 10 mcg/ml (100 mcg/ml) | 10:1 |
| ig. adm | 67 mg/kg (190 mg/kg) | 134:1 (380:1) | 100 mcg/ml (1000 mcg/ml) | 100:1 |

As it can be observed in FIGS. 2, 3, and 4, the desensitization to the Capsaicin increases from the iv. level to ig. level. The Capsaicin is more insensitive in stomach than in blood. This phenomenon is in agreement with the researches of Franco et al. (1979) and Holzer et al. (1980), already mentioned. Values of desensitization scale increase because more substance p(sp) is released, thus an injected organism with Capsaicin gets newer analgesics stage and sensitivity to the Capsaicin decreases.

Concentrations

Concentrations increase by following to the FIGS. 2, 3 and 4. Thus recommended concentrations of Capsaicin to apply according to physician criterial for iv., im./sc. and ig. treatments are:

| | CONCENTRATION | | CONCENTRATION | | CONCENTRATION |
|---|---|---|---|---|---|
| No iv. | (mcg/ml) | No im./sc. | (mcg/ml) | No ig. | (mcg/gr) |
| 1 | 2.0 | 1 | 10 | 1 | 100 |
| 2 | 2.5 | 2 | 15 | 2 | 125 |
| 3 | 3.0 | 3 | 20 | 3 | 150 |
| 4 | 3.5 | 4 | 25 | 4 | 175 |
| 5 | 4.0 | 5 | 30 | 5 | 200 |
| 6 | 4.5 | 6 | 35 | 6 | 225 |
| 7 | 5.0 | 7 | 40 | 7 | 250 |
| 8 | 5.5 | 8 | 45 | 8 | 275 |
| 9 | 6.0 | 9 | 50 | 9 | 300 |
| 10 | 6.5 | 10 | 55 | 10 | 325 |
| 11 | 7.0 | 11 | 60 | 11 | 350 |
| 12 | 7.5 | 12 | 65 | 12 | 375 |
| 13 | 8.0 | 13 | 70 | 13 | 400 |
| 14 | — | 14 | 75 | 14 | 425 |
| 15 | — | 15 | 80 | 15 | 450 |

Dosage

Szolcsanyi et al. (1975), p. 1878, researched Capsaicin potency as an alkaloid, among fifty (50) spicy congeners and derivatives, they found that Capsaicin, is by far the strongest pungent agent. Toxicity of Capsaicin is exalted at high doses (mg/kg), and inhibited at low doses rising its therapeutic faculties.

According to Monsereenusom (1983), p. 106, in some tropical countries (India and Thailand) the daily intake of capsicum fruit is between 0.5-1.0 mg/kg body weight.

These doses consumed in large quantities can be considered as maximum doses at digestive level. In agreement with the potency of Capsaicin, doses between 0.5-1.0 mg/kg at digestive level are equivalent to doses between 5-10 mcg/kg at intravenous level.

The Capsaicin dosage for iv. and im./sc. treatment will oscillate between 1 mcg/kg and 7-8 mcg/kg. for the ig. treatment the dosage will oscillate between 100 mcg/kg and 450 mcg/kg. doses for iv. and im./sc. treatments are equals because of the feasible volumes to be infused.

A standard dosage for iv., im./sc. and ig. treatments it is as follows:

| | DOSES | | DOSES | | DOSES |
|---|---|---|---|---|---|
| No iv. | (mcg/kg) | No im./sc. | (mcg/kg) | No ig. | (mcg/kg) |
| 1 | 1 | 1 | 1 | 1 | 100 |
| 2 | 1.5 | 2 | 1.5 | 2 | 125 |
| 3 | 2 | 3 | 2 | 3 | 150 |
| 4 | 2.5 | 4 | 2.5 | 4 | 175 |
| 5 | 3 | 5 | 3 | 5 | 200 |
| 6 | 3.5 | 6 | 3.5 | 6 | 225 |
| 7 | 4 | 7 | 4 | 7 | 250 |
| 8 | 4.5 | 8 | 4.5 | 8 | 275 |
| 9 | 5 | 9 | 5 | 9 | 300 |
| 10 | 5.5 | 10 | 5.5 | 10 | 325 |
| 11 | 6 | 11 | 6 | 11 | 350 |
| 12 | 6.5 | 12 | 6.5 | 12 | 375 |
| 13 | 7 | 13 | 7 | 13 | 400 |
| 14 | — | 14 | 7.5 | 14 | 425 |
| 15 | — | 15 | 8 | 15 | 450 |

Obtaining of the Capsaicin and Preparation of the Infuses.

To obtain the Capsaicin for a preparation of the infuses, the substance will be requested from Sigma-Aldrich laboratories, located in St Louis, Mo., USA. These laboratories produce Capsaicin with 97% of purity.

A procedure to obtain Capsaicin according to a standard technique it uses fresh chiles which are sliced, dried in a tray with air at 140 degrees fahrenheit, and ground to pass through a thirty-mesh screen. The powder is treated with a solvent; for a water-soluble preparation, ethanol is the usual solvent. Residual materials are eliminated. After this procedure is complete, the solvent is removed from Capsaicinoids by distillation or by any of a known conventional method.

The infuses will contain Capsaicin, with the recommended concentrations, 5-10% or less of ethanol to dilute Capsaicin; 5-10% of polyoxyenthylenesorbitan monooleate also called tween.rtm.80 to be used as emulsifier, surfactant, stabilizer and dispersant; and distilled water to complete a volume of infuses. The infuses also can be prepared with saline isotonic solution with 0.90% of sodium chloride (Nacl) instead distilled water.

Capsaicin as was above pointed out is more soluble in alcohol and oils than water.

Preparations of the infuses must be done with a guarantee that they will have the recommended concentrations. For it, the infuses will be submitted to a test for a verification of the Capsaicin concentration required, which it can be carried out by hplc and using a spectrofluorimeter.

Capsaicin iv. and im./sc. administration will be carried out through of a maximum number of 13 high volume infuses (hvi), and 15 low volume infuses (lvi). lvi increase its volume until 40r 5 ml, while that hvi increase its volume until 75 ml. Capsaicin ig. administration will be supervised through a consumption of capsicum sp fruits or ingestion of capsules.

### Example of a Preferred Embodiment

Preparation of one liter and more of the first infuse at the concentration of 2 mcg/ml of Capsaicin for the iv. treatment is made by mixing ethanol, tween.rtm.80, and saline isotonic solution to obtain the following proportions of a pharmaceutical acceptable carrier (v/v):

5% ethanol (diluent)

10% tween.rtm.80 (emulsifier)

85% saline isotonic solution (second diluent)

Procedure to Prepare the Preferred Embodiment.

1. Elaboration of a Capsaicin prime soluton by using an amount of 1.0 g of Capsaicin 97% purity (molecular weight 305.4) and 50 ml of ethanol 95%, which are mixed until Capsaicin is disolved. 100 ml of tween.rtm.80 are added to such solution until Capsaicin is evenly dispersed. 850 ml of distilled water are mixed to the prime solution, which it is agitated. This resulting prime solution will contain a concentration at 1 mg/ml. of Capsaicin.

2. Elaboration of a carrier solution without Capsaicin by mixing 50 ml of ethanol, 100 ml of tween.rtm.80 and 850 ml of saline isotonic solution which it is agitated.

3. From the prime solution it is extracted 3 ml, being it diluted in 997 ml of the carrier solution. This infuse solution will approximately contain 3 mcg/ml.

4. The infuse solution is submitted to a test of hplc to determine a concentration of Capsaicin.

5. After of knowing such exact concentration, a volume of the infuse solution is increased by using the carrier solution to rectify the concentration at 2 mcg/ml. thus it is obtained a final infuse solution.

6. From that final infuse solution it is extracted a required volume according to the first dose for the iv. treatment at 1 mcg/kg of bodyweight.

7. For a 50 kg-patient it is necessary a infuse volume of 25 ml (6.5 mcm) to be injected by iv. administration without anesthesia. thus one HIV patient will feel during such infused injection a sensation of perceptible warmth.

Capsicum Extract Infuses

Capsicum extract has a bigger power of healing than Capsaicin owing to several compounds contained in the extract that act by synergetic mechanisms. Other natural chemicals compounds founded in the extract such as piperine, piperidine, piperyline, piperettine, piperolein a and b, and piperanine have a minor degree of pungency than Capsaicin and dihydroCapsaicin. This method intent to determine a total degree of pungency of the extract in scoville units (su) for establishing an equivalence with a degree of pungency of Capsaicin infuses in iv. and im./sc. treatments. According to the National Institute of Justice (1995), p. 11, table 4, in the Preliminary investigation of oleoresin capsicum, samples with Capsaicin and compounds related, analized by liquid chromatography with mass spectometric detection, shows Capsaicinoids concentration at (mg/g) and its (su) at (ml/g). This inforamation permitted to calculate (su) of Capsaicinoids for 1 mcg/ml (1 ppm). It resulted at (16 su). DeWitt (1999), p. 244, confirms it in about 30 su for 2 mcg/ml (2 ppm) of Capsaicin.

A general procedure covers the following steps:

1. Approximately 100 gr of fresh plant tissues of chilies are washed and soaked in distilled water and being dried.

2. Fruits are then chopped and ground intermittently in a blender until a homogenous liquid and solid material is obtained.

3. This material is strained to discard solid matter.

4. The filtrate is centrifuged at 4 degrees centigrade for 30 min. the supernatant is collected and recentrifuged.

5. the final supernatant is tested by hplc to determine pungency and concentration of Capsaicinoids and other chemical compounds and estimate the total and general pungency of the extract. The hplc process dissolves a sample in ethanol saturated with sodium acetate and sparates out chemical compounds, which they are analized with a spectrofluorimeter that measures the concentration in parts per million (ppm), which it is then converted to (su).

6. Sterilize by heating the extract inside a large test tube, which may be placed into a boiling water bath. The extract is swirled every 5 min and placed on ice or in refrigeration conditions after 20 min of heating.

7. A carrier solution is prepared by containing just diluents as above mentioned for Capsaicin infuses preparation.

8. Diluting an aliquot of the extract by using the carrier solution until 32 (su), which it is the pungency of the iv. first infuse. It make possible the equivalence referred.

9. Preserve capsicum extract infuses after they are prepared in sealed containers and refrigeration conditions.

Steps 1 to 4 were taken from the method of Cichewicz et al. 1996, p.62.

Non-Toxic Effect of tween.rtm.80 and Ethanol

Because of a use of tween.rtm.80 as a dispersant for the medication in this invention, it is herein mentioned in order to determine its possible toxic effect by an iv. or im./sc. treatment. tween 80 was evaluated for potential toxicity in a report of 1992 denominated, developmental toxicology of polyoxyethylene sorbitan monooleate (cas # 9005-65-6) in Sprague-Dawley cd rats, p. 1, abstract. in such experiment, pregnant rats were exposed to 0, 500 or 5000 mg/kg/day of tw80, concluding that, no dose-related signs of toxicity were observed for individual animals during the in-life phase of the study or at scheduled necropsy.

Likewise, ethanol has a toxic effect according to the amount that is absorbed or comsumed. In agreement to a report, chemical of the week-ethanol, pp. 1-2, over 90% of it is processed by the liver in the liver, the alcohol dehydrogenase enzyme converts ethanol into acetaldehyde which is destroyed almost immediately by the aldehyde dehydrogenase enzyme, which converts it to acetate ions. 1 ounce shot of 100-proof whiskey, which contains 0.5 fluid ounces of ethanol (about 15 ml), is diluted 5000-fold in a 150 pound human, producing a 0.02% blood alcohol concentration. A 0.05% it starts to produce a measurable mental impairment.

An infuse of 25 ml as calculated in the preferred embodiment only contains an amount of 1.25 ml of ethanol if it is introduced a concentration of 5% of ethanol in the pharmaceutical carrier. It is not considered risky and significant.

Duration of the Treatment

The iv. and the im./sc. administration will have a duration of 26 days for i.v. treatment and 30 days for im./sc. treatment. Each dose will be administered leaving to the patient, at least one day off to rest between two doses. The treatment must be handled in opening and flexible form, according to a medical criterial and side effects of the Capsaicin.

Frequency of the Treatment

After a patient has received the Capsaicin administration by a period of a month, must be left in resting at least for 30 days consecutive. In agreement with Monsereenusorn et al. (1982), p. 329, the recovery of the desensitization is uniformly slow and it is not complete even after 60 days. This assertion mentioned, is pertinent for Capsaicin administration at high concentration.

Side Effects

Some side effects of the Capsaicin, as much as by iv. and im./sc. administration are: sweating, moderated decrease in levels of glucose in blood, light decrease in corporal temperature, headaches and nasal secretion like a product of histamine increase, and momentary decrease in cardiac rate and arterial pressure. It is esteemed that such effects will be attenuated by applying the pretreatment.

However, during an implementation of the treatment is necessary a constant inspection and examination of: temperature, arterial pressure, cardiac rate, level of glucose, histamine and consumption of liquids by tee patient. Risks of the iv. treatment are minimum, in fact letal dose in rats is given at 500 mcg/kg (0.5 mg/kg). In this invention a maximum dose is around 7-8 mcg/kg. Although the risk is minimum, atropine is considered a substance which it is able to counteract the effect of Capsaicin (Chahl et al. 1987, pp. 414-415).

Some side Capsaicin effects are considered beneficial to control HIV. Light hypothermia and hypotension are factors to inhibit HIV replication. Light toxicity at low Capsaicin doses and concentrations is a factor to activate immunization mechanisms. Furthermore, momentary changes in cardiac rate and arterial pressure are factors of a sanguineous stimulation, DeWitt (1998).

Operation of the Treatment

The operation of the treatment system for HIV, will mainly depend on the patients conditions and on the advance stage of the illness. Patients that have been detected with the illness in its early or primary phase can be submitted to the iv. treatment, applying previously the pretreatment with Capsaicin. Patients that have been detected in its intermediate phase or asymptomatic period can be submitted to the im./sc. treatment. Likewise, patients detected in the final phase or full blown must be initially submitted to the ig. treatment.

The following program of treatments has been designed according to the stage of HIV on each patient, and it shows a sequence of treatments to follow:

| TREATMENTS | PRIMARY PHASE | ASYMPTOMATIC PERIOD | FULL BLOWN |
|---|---|---|---|
| 1 | PRETREATMENT | im./sc. ADMIN | ig. ADMIN |
| 2 | iv. ADMIN | PRETREATMENT | im./sc. ADMIN |
| 3 | — | iv. ADMIN | PRETREATMENT |
| 4 | — | — | iv. ADMIN |

During the administration of the substance must observed, once injected the infuses, the effects reflected by the patient. The inicial doses will produce certain effects, and increment of the doses will produce an attenuation of such effects because desensitization to the Capsaicin is provoked.

Patients that have been detected in the final phase of the illness, must be submitted to the ig. treatment to counteract opportunistic illnesses related to the digestive system, and later to the im./sc. treatment. Finally the physician can opt for the iv. treatment. This procedure also can be applied to patients detected in the intermediate phase.

Capsaicin concentration applied at its lowest level it will not require previous anesthesia in any of treatments because a sensation felt by patient at this concentration it is defined like perceptible warmth. An use of anesthesia it will distort a treatment in relation to a sudden and inadequate release of neuropeptides.

The im./sc. treatment does not require the pretreatment with Capsaicin.

To estimate how the treatment with Capsaicin controls HIV, is necessary to use a method for monitoring the effectiveness of Capsaicin in each patient through a test of lymphocytes t4. By this way the physician can observe a clinical progression of HIV infection and its response to the Capsaicin, to make decisions in the treatment.

Macrophages Proliferation Control

It is important to get support about a method of modulation of macrophage proliferation, because Capsaicin iv. treatment may increase proliferation of macrophages and phagocytosis.

A control of macrophage proliferation has been considered in a foreign patent number WO 99/21542° f. Mcgrath, 1999, methods for modulating macrophage proliferation using polyamine analogs.

According to McGrath, 1999, results of an effect of one polyamine analog (dehop) on proliferating macrophages from the blood of four patients with AIDS dementia are shown in p. 1 and pp. 28-300f the invention, FIG. 1A, table 3. This polyamine analog significantly reduced the percentage of macrophages between 19 and 97% using concentrations that varied between 0.08 and 50 mcm. Other polyamine analogs such as Denop, BE-4444, SL-11037, SL-11038, SL-11048, SL-11047, SL-11044 showed a potent effect of inhibition (see table 2, FIGS. 1b, 5, and 6).

In agreement to the invention of McGrath, p. 25, these polyamine analogs may be administered by subcutaneous or intravenous injection but they may also be administered orally. Doses generally range between 1 to about 300 mg/m2/day, and possibly between 15 to about 150 mg/m2/day. Administration of such medications is generally intermittent being prescribed per a period of at least one to two days and not administered for a same period. Such pharmaceutical medications are manufactured by SunPharm and S'lil Pharmaceuticals.

A proliferation of macrophages must be monitored and quantified during or after a treatment with Capsaicin to prescribe according to physician criterial polyamine analogs by considering the specifications of the mentioned invention.

Infuses Operation

Capsaicin treatment with injected infuses for HIV patients brings with itself risks of contamination of the medical personnel, handling syringe and tools. Nevertheless by using kevlar gloves, sleeves and aprons assure a right procedure avoiding needle stick injuries. This material has a high breaking strength, toughness and cut resistance. Thus can be used kevlar lightweight material, or another customized type for medical use specifically during a beginning of the treatment to implant an iv. device and after a final dose to remove it. When it is used a non-implantable device, gloves must be used throughout the course of any treatment. Such gloves must be fabricated with external plastic aseptic material to make sure sterility is maintained.

To make safer administration of Capsaicin in HIV patients without a continuous requirements of handling syringe, have been designed safe needleless connecting devices to administer infuses. They consist of a blunt-tipped insertion device and a corresponding rubber injection port with a slit that opens and reseals immediately. It also must be mentioned that infuser pumps control the velocity of administration of the medication.

For an iv. device safe implanting may be considered to use an ultrasound scanner optimized for vascular imaging and configured for needle guidance and vascular access, manufactured by bard access system. It may also be used a venoscope transilluminator developed by Applied Biotech Products, inc. They allow the clinician to see a vessel, observe a needle puncture and place a line by safe manner.

Two of the most important complications of implanting intravenous devices are thrombosis, and local infections as a result of deficient asepsis. Such complications are attenuated by Capsaicin, if it is considered its antimicrobial characteristics already mentioned and its anti-clots properties. Visudhiphan et al. (1982), p. 1452, suggested to capsicum as protection against blood clots by causing an increase in fibrinolytic activity.

### Conclusions

Mayan civilization left very well established the healing powers of capsicum. Actually Capsaicin being domesticated under specifications of this invention may be proyected as a future generic antivirus.

It well be apparent to those skilled in the art that modifications can be made without departing from the object and scope of the present invention. Therefore, it is intended that the invention only be limited by the claims.

What is claimed is:

1. A method of medical treatment of devastating effects against a human immunodeficiency virus and a acquired immunodeficiency syndrome by using a medication with any of a natural and synthetic Capsaicin, related derivatives, vanilloids and capsicum extract comprising:
(a) providing an intravenous, intramuscular/subcutaneous and digestive treatments with said medication, and
(b) providing an intramuscular/subcutaneous pretreatment previous to said intravenous treatment making permissible an administration of said medication by this way in a human body, and
(c) providing an independent and combined administration of the substance, through a periodic and subsequent application of said treatments means according to a different stage of said diseases in a patient, and
whereby said intravenous treatment will produce, a neuropeptides realease in humans, stimulating it a immune system, a virus replication inhibition and a control of opportunistic diseases related, and
whereby said intramuscular/subcutaneous treatment will serve as an alternative treatment to said intravenous treatment, and
whereby said intramuscular/subcutaneous pretreatment will minimize a Bezold-Jarisch reflex by reducing a side effect of said medication, and
whereby said digestive treatment will provoke an antioxidant effect and it will also help to a control of opportunistic diseases related, specially it that attacks a digestive system.

2. A method of medical treatment of claim 1, wherein said treatments are applied, comprising:
(a) administration of said medication following it a pattern according to a function of values of x equal to 1/x, where a value of x is bigger than 0, in agreement to FIGS. 2, 3 and 4, and
(b) administration of said medication, wherein said treatments are equivalents by means a potency of the substance, and
(c) administration of said medication following said function and a predetermined range of concentrations and doses defined upon an aside of concentrations and dosage, and
(d) administration of said medication, wherein said predetermined range of concentrations is limited between a concentration that provokes a sensation of perceptible warmth and a concentration not reaching it a sensation of persistent burning, and
(e) administration of said medication by increasing a dose and a concentration in said predetermined range, means for inducing a light nervous desensitization process in the human organism, and
(f) administration of said medication by regulating a velocity of injecting at least at 1 ml/min for a first half of medications and at 1 ml/30 sec for a remainder of medications.

3. A method of medical treatment of claim 2, wherein said predetermined range of concentrations and doses is defined comprising:
(a) providing an intravenous administration of said medication, through a range of concentrations between 2 mcg/ml and 8 mcg/ml, and a range of doses between 1 mcg/kg and 7 mcg/kg of body weight, and
(b) providing an intramuscular/subcutaneous administration of said medication, through a range of concentrations between 10 mcg/ml and 80 mcg/ml, and a range of doses between 1 mcg/kg and 8 mcg/kg of bodyweight, and
(c) providing a digestive administration of said medication, through a range of concentrations between 100 mcg/gr and 450 mcg/gr, and a range of doses between 100 mcg/kg and 450 mcg/kg of bodyweight.

4. A method of medical treatment of claim 1, wherein said treatments are applied by leaving a patient period of resting comprising:
(a) of at least thirty consecutive days among said treatments, and
(b) of at least a day off among two different doses.

5. A method of medical treatment of claim 1, where said treatments are applied, comprising:
(a) a duration of twenty six days for said intravenous treatment by applying a maximum of thirteen doses, and
(b) a duration of thirty days for said intramuscular/subcutaneous and digestive treatments by applying a maximum of fifteen doses in each of one, and
(c) a duration of three days for said intramuscular/subcutaneous pretreatment.

6. A method of medical treatment of claim 1, wherein an administration of said medication for the intramuscular/subcutaneous pretreatment is defined, comprising:
(a) providing a concentration and dose of said medication which they are equivalents respectively to a first concentration and dose of said intravenous treatment, and
(b) providing the administration of said medication being defined for a total dose at 10 mcg/kg of body weight, which is ten times bigger than said first dose of said intravenous treatment, which is at 1 mcg/kg of the bodyweight, and
(c) extending the administration of said medication on a same injecting area to apply said intravenous treatment by administering, 2 mcg/kg of bodyweight in a first day, 3 mcg/kg in a second day and 5 mcg/kg in a third day, and
(d) providing the administration of said medication by applying a concentration at 20 mcg/ml in the first day, 30 mcg/ml in the second day and 50 mcg/ml in the third day.

7. A method of medical treatment of claim 1, further including administration of said medication by topical treatment and systemic effect of absorption, using same doses and concentrations of said intramuscular and subcutaneous treatment as shown upon the aside of concentrations and doses.

8. A method of medical treatment of claim 1, wherein said immune system stimulation and a effectiveness of the substance is monitored through a test of lymphocytes t4 to observe a clinical progression of said diseases and its response to said concentrations and doses of the medication.

9. A fabrication and use of a medication by including an active substance such as any a natural and synthetic Capsaicin, related derivative, vanilloid compound and capsicum extract against a human immunodeficiency virus and a acquired immunodeficiency syndrome, comprising:
(a) fabrication and use of said medication by including any of said natural Capsaicin and related derivatives, according to a chemical formulate of each one, showed in the FIGS. 1a, 1b, 1c, 1d, 1e, and
(b) fabrication and use of said medication by including any of said synthetic Capsaicin and vanilloid compounds mentioned in a definition of terms of the invention, and
(c) fabrication and use of a pharmaceutical carrier solution containing, a alcohol denominated ethanol ranging it between 5 and 10% v/v as preferred diluent, and said alcohol with less than 5% v/v as more preferred diluent, and
(d) fabrication and use of said pharmaceutical carrier solution by adding a emulsifier and stabilizer denominated polyoxyenthylenesorbitan monooleate, also called tween.rtm.80 ranging it between 5 and 10% v/v, and
(e) fabrication and use of said pharmaceutical carrier solution by finally adding to complete a volume of the medication with a distilled water as preferred second diluent, and a saline isotonic solution with 0.9% of sodium chloride as more preferred second diluent, and
(f) fabrication and use of said medication by elaborating an infuse of high volume for a intravenous treatment, and an infuse of low volume for a intramuscular/subcutaneous treatment and intramuscular/subcutaneous pretreatment, and
(g) fabrication of said infuses by following as example a procedure to manufacture a first intravenous infuse exposed upon a aside of a preparation of a preferred embodiment.

10. A fabrication and use of the medication of claim 9, wherein said procedure to manufacture an intravenous first infuse is taken as example to elaborate a rest of infuses comprising:
(a) elaboration of 1 liter of a prime solution by using a amount of 1 gr of any of said natural and synthetic Capsaicin, related derivative and vanilloid, and 5% v/v of said diluent, which are mixed until said active substance is disolved obtaining a solution which it is agitated, and
(b) adding a amount of 10% v/v of said emulsifier to said solution until the active principle is evenly dispersed, and adding 85% v/v of a distilled water to complete a volume of the resulting prime solution which it is agitated, and
(c) elaboration of a carrier solution by mixing 5% v/v of said diluent, 10% v/v of said emulsifier and 85% v/v of said second diluent which it is agitated, and
(d) taking 3 ml of said resulting prime solution and diluting it in 997 ml of said carrier solution to obtain a concentration approximately at 3 mcg/ml and submitting such an infuse solution to a test of high performance liquid chromatography to determine and verify a exact concentration, and
(e) increasing a volume of said infuse solution by using said carrier solution to rectify a concentration at 2 mcg/ml obtaining a final infuse solution, and
(f) extracting a required volume from said final infuse solution according to an intravenous first dose at 1 mcg/kg bodyweight of a patient, and
(g) fabricating a remainder of the intravenous and intramuscular/subcutaneous infuses by following said same procedure, but considering concentrations and doses of said infuses referred upon a aside of concentrations and doses of the invention.

11. A fabrication and use of said medication of claim 9, further including a procedure to manufacture said capsicum extract infuses comprising:
(a) taking 100 gr of a fresh plant tissue of chilies, which are washed and soaked in a distilled water, and being dried, and
(b) chopping and ground intermittently said fresh plant tissue of chilies in a blender until a homogenous liquid and solid material is obtained, and
(c) straining said homogenous liquid and solid material to discard said solid material and obtain a filtrate, and
(d) centrifuging at 4 degree centigrades for 30 min said filtrate to obtain a supernatant, and
(e) collecting and recentrifuging said supernatant to obtain a final supernatant, and
(f) testing by high performance liquid chromatography said final supernatant to determine a general pungency in scoville units, and
(g) sterilizing by heating said final supernatant inside a large test tube, which may be placed into a boiling water bath, being swirled every 5 min and refrigerated after 20 min of heating, and
(h) fabricating a carrier solution which it is elaborated by containing just diluents as mentioned upon the aside of a procedure to prepare a preferred embodiment for Capsaicin infuses, and
(i) diluting a aliquot of said final supernatant by using said carrier solution until 32 scoville units for every 2 mcg/ml of said Capsaicin contained in intravenous and intramuscular/subcutaneous infuses to prepare, and
(j) preserving infuses after they are prepared in sealed containers and refrigeration conditions.
